(19) United States
(12) Patent Application Publication
(10) Pub. No.: US 2021/0161174 A1
(43) Pub. Date: Jun. 3, 2021

(54) FEED ADDITIVE FOR FARMED ANIMALS

(71) Applicant: ADISSEO FRANCE S.A.S., Antony (FR)

(72) Inventor: Pierre-André GERAERT, Rochecorbon (FR)

(21) Appl. No.: 17/047,824

(22) PCT Filed: Apr. 19, 2019
(86) PCT No.: PCT/IB2019/000520

§ 371 Date: Oct. 15, 2020

Related U.S. Application Data
(60) Provisional application No. 62/660,456, filed on Apr. 20, 2018.

(51) Int. Cl. A23K 20/132; A23K 50/75

(52) U.S. Cl. CPC A23K 20/132; A23K 50/75

(57) ABSTRACT

A use of pyrroloquinoline quinone (PQQ) for at least one of preserving the quality of meat obtained from farmed animals, relates to preventing the apparition of the defaults in meat, limiting, stopping the development of defaults in meat. The defaults are linked to myopathy, with farmed animals, and preparing a feed additive or a premix in order to preserve the quality of meat obtained from farmed animals and/or in order to at least one of preventing the apparition of the defaults in meat, limiting and stopping the development of defaults in meat. The defaults are linked to myopathy, with farmed animals. The use also relates to feed additive, a feed and a premix having PQQ.

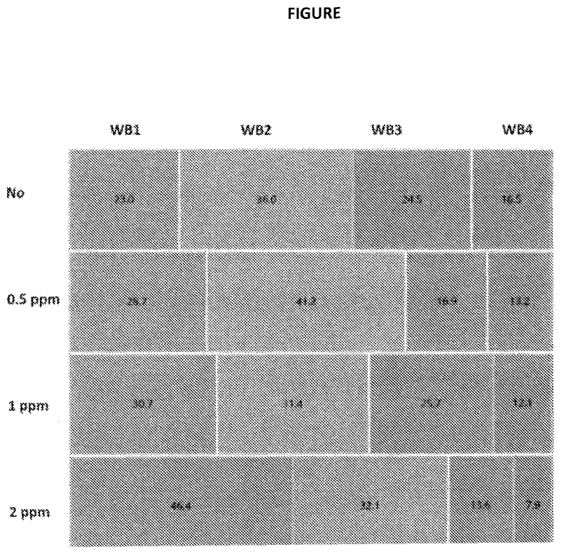

### TECHNICAL FIELD

The present disclosure relates to feed for farmed animals, and specially a feed additive that improves the quality of the meat which is obtained from them.

### BACKGROUND

Several studies have reported the outbreak of meat defaults linked to myopathies in animals, in particular in animals whose growth and meat yields are the highest, having negative consequences on the meat quality. This has been observed with the main farmed species (pig, cattle, sheep, poultry, fish) (Lebret et al., 2015, INRA Prod. Anim., 28, 151-168).

In swine, destructured ham is a technological defect of great importance due to the economic loss it can cause. It has been studied from the biochemical perspective to tissular characterization and appeared related to myopathies (Theron et al., 2019, Food Chem., 70, 359-366).

Poultry are affected by two main breast myopathies, White Striping (WS) and Woody Breast (WB), and these diseases are of concern to poultry meat producers as they have undesirable consequences on the fillets.

These abnormalities which are grouped so far under the term “myopathies” have been reported to be linked to rapid muscle growth that could affect the development of the muscle tissue of the animals. This has a huge negative impact on the appearance and quality of the meat, and consequently on consumption of the affected meat pieces.

Hence, a lot of research is presently ongoing to find a solution, but to date, no effective response has been given, whereas there is an urgent need to overcome this difficulty.

In poultry, White Striping is visually evidenced by white striations parallel to the axis of the muscle fibers. The appearance of the product is degraded appearing fatter, with lower protein content, and is rejected by the consumers (Kuttapan et al., 2013, Poultry Science, 92, 811-819). These breasts also exhibit a lower water holding capacity, higher cooking losses reducing the technological yield and a reduced firmness than normal fillets.

Wooden Breast shows variation in coloration of the meat, an excess in drip loss on the surface of the breast and a loss of elasticity as well as hardness and tough areas (Sihvo et al., 2014, Veterinary Pathology 51(3):619-623). Moreover, these breasts have poor technological properties impairing their use for further processing.

A recent survey showed that in French slaughter plants, even the standard or semi-heavy broilers (1.9 to 2.3 kg) are affected by these breast defaults. White Striping appeared in all batches with an average 33 to 90% birds affected. Wooden Breast is detected in 98% of the flocks affecting 10 to 70% of the breasts. Another breast myopathy, spaghetti meat, could be detected in 65% of the flocks with up to 20% of the birds. More than one default can also be detected in all flocks. In Italy, a similar survey showed WS between 70 to 82% in standard and heavy broilers with 13 to 26% birds showing severe defaults (Russo et al., 2015). In experimental conditions, 50 to 80% defaults can occur. The economical loss can reach 200 M$ per year in USA only (Kuttapan et al., Poult Sci., 2017, 96(8) 3005-3009).

With such an occurrence, most of the breeders will face breast myopathies in all types of chickens even below 2 kg live weight in less than 5 years. Slowering growth through nutritional dilution appeared reducing WB and WS but with a strong degradation of the efficacy of the production. Moreover, the direct nutritional enrichment in antioxidants, vitamin E, selenium, organic minerals has not shown significant benefits.

The economic consequences are variable with reducing the use of fast growing high breast yield chickens, withdrawal of the affected birds by vet services in the slaughter plants, diverting the meat for further processing usage with reduced technological yield. Moreover, these defaults could affect bird welfare and could reduce nutritional value of the meat. Lastly, they could jeopardize the healthy image of the poultry meat.

Thus developing an efficient solution to reduce breast defaults is key for the poultry industry and its sustainability.

### SUMMARY

The purpose of the present disclosure is to provide a solution to this problem. In accordance with the disclosure, this problem is solved by supplementing the animal feed with a compound which is readily available, in particular it is a naturally-occurring compound, and has no other impacts on the animals or on the meat therefrom than solving the above problem.

The present disclosure will be exposed and detailed in reference to preventing or limiting the abnormalities above-mentioned, with poultry. But, as indicated above, it extends to any farmed animals exhibiting meat defaults that could be linked to myopathy, or to any farmed animal that could be undergoing a myopathy, where said myopathy is or could be associated with a negative effect on the quality of at least certain cuts of meat from these animals.

The present disclosure provides a remedy for preventing the inconveniences resulting from myopathies in farmed animals.

In an embodiment of the disclosure, this solution aims at preventing breast defaults linked to breast myopathies in poultry. In a further embodiment, it aims at preventing wooden breast and/or white stripping in poultry.

According to the disclosure, it has been surprisingly found that pyrroloquinoline quinone (PQQ) when fed to a farmed animal is able to prevent the apparition of meat defaults linked to myopathy, or is able at least to limit, and even to stop, the developments of the defaults in meat linked to myopathy, even with animals that are prone to such disorder, for example with animals whose growth and meat yields are the highest.

In the context of the disclosure, this solution aims at preventing or treating breast myopathies in poultry and the disclosure pertains to the following subjects:

a method for preventing or treating myopathies in poultry, in particular for preventing or treating at least one of wooden breast, white stripping and spaghetti meat, and in an embodiment of the disclosure, the method comprises administering pyrroloquinoline quinone (PQQ) or salt thereof to said poultry,

a feed additive for preventing or treating breast myopathies in poultry, said feed additive comprising pyrroloquinoline quinone (PQQ), and in an embodiment of the disclosure, the feed additive is for preventing or treating at least one of wooden breast, white stripping and spaghetti meat, and

a feed or a premix comprising PQQ or a feed additive comprising PQQ, in the indication above.

In accordance with a main embodiment, the purpose of this disclosure is to prevent the apparition of the defaults in meat, or to limit or even to stop the development of defaults in meat, said defaults being linked to myopathy, in any farmed animals, such as poultry, pig, cattle, sheep, fish. In this context, the disclosure relates to the following subjects:

use of pyrroloquinoline quinone (PQQ) for preserving the quality of meat obtained from farmed animals;

use of pyrroloquinoline quinone (PQQ) in at least one of preventing the apparition of the defaults in meat, limiting and stopping the development of defaults in meat, said defaults being linked to myopathy, with farmed animals;

use of pyrroloquinoline quinone (PQQ) for preparing a feed additive, a feed or a premix in order to preserve the quality of meat obtained from farmed animals;

use of pyrroloquinoline quinone (PQQ) for preparing a feed additive, a feed or a premix for at least one of preventing the apparition of the defaults in meat, limiting and stopping the development of defaults in meat, said defaults being linked to myopathy, with farmed animals;

a feed additive for preserving the quality of meat obtained from farmed animals, said feed additive comprising pyrroloquinoline quinone (PQQ);

a feed additive for at least one of preventing the apparition of the defaults in meat, limiting and stopping the development of defaults in meat, said defaults being linked to myopathy, with farmed animals, said feed additive comprising pyrroloquinoline quinone (PQQ);

a feed or premix for preserving the quality of meat obtained from farmed animals, said feed or premix comprising pyrroloquinoline quinone (PQQ) or a feed additive of the disclosure;

a feed or premix for at least one of preventing the apparition of the defaults in meat, limiting and stopping the development of defaults in meat, said defaults being linked to myopathy, with farmed animals, said feed or premix comprising pyrroloquinoline quinone (PQQ) or a feed additive of the disclosure.

According to the disclosure, the meat is protected from the adverse effects of myopathies as previously described. In one embodiment, said farmed animals are prone to or affected by myopathy, wherein said myopathy causes defaults in the meat obtained from said farmed animals.

The present disclosure also provides a method for preventing the apparition of the defaults in meat, or to limit or even to stop the development of defaults in meat, said defaults being linked to myopathy preventing, with farmed animals; in an embodiment of the disclosure, the method comprises administering pyrroloquinoline quinone (PQQ) to said animals; in a further embodiment of the disclosure, the method comprises administering a feed additive comprising PQQ to said animals. In other embodiment, it comprises feeding the animals with a feed or a premix in accordance with the disclosure. Preferably, said farmed animals are prone to or affected by myopathy wherein said myopathy causes defaults in the meat obtained from said farmed animals.

PQQ which formula is given below, also known as methoxatin, is a cofactor of a number of bacterial deshydrogenases and is therefore naturally present in plants, such soybean, parsley, green pepper, green tea, papaya, kiwi. It has not been demonstrated to be biosynthesized in mammals, and its presence in higher organisms is probably from microbial origin. Thus, PQQ is found in milk. However, its concentrations in natural sources are generally very low and it is commercially available by chemical synthesis (among others WO2014/195596A1) or by fermentation process (Mitsubishi Gas Chemical Company Inc).

Formula of PQQ:

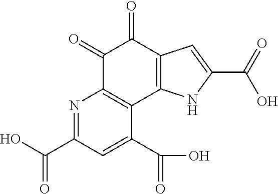 [SMILES: O=C(O)c1cc(C(=O)O)c2c(n1)C(=O)C(=O)c1cc(C(=O)O)[nH]c1-2]

In accordance with the disclosure, in any embodiment, the PQQ may be in the form of a salt, for example a sodium or potassium salt.

The PQQ may be from natural source or prepared via fermentation or chemical synthesis. When PQQ is from natural source, it may be extracted from a natural source, it may also be provided in the form of an extract that may be concentrated in PQQ or by the compounds naturally comprising PQQ, after an optional treatment. Accordingly, the disclosure extends to any use as described above, of an extract which naturally comprises PQQ, said extract being treated or not. Advantageously, the extract is treated to increase its PQQ concentration. This treatment is carried out by any physical or chemical method, which belongs to the skilled in the art's common knowledge.

In one embodiment, PQQ is liquid or granular. Whatever the presentation of PQQ, It may be formulated with additional components such as surfactants, binding agents, water.

The administration of PQQ is generally carried out by ingestion, said PQQ being mixed in feed or in drinking water of farmed animals. In a preferred embodiment, PQQ is in the form of granules that are mixed with a premix or a feed.

In accordance with the above mentioned subjects of the disclosure, PQQ or a salt thereof is used in an effective amount for preventing myopathy. This amount is determined on the basis of the farmed animal species and if details are given below for poultry, the skilled in the art is capable to determine an appropriate amount of PQQ or a salt thereof.

The present disclosure is now exposed in more details in the context of meat defaults linked to myopathies in poultry.

Thus, in accordance with other embodiments, the present disclosure relates to the following subjects:

pyrroloquinoline quinone (PQQ) for use in preventing myopathies in poultry, such as breast myopathy, and in particular at least one of wooden breast, white stripping and spaghetti meat;

a feed additive for use for preventing myopathies in poultry, such as breast myopathy, and in particular at least one of wooden breast, white stripping and spaghetti meat, said feed additive comprising PQQ;

use of PQQ for preserving the quality of meat obtained from poultry or to the use of PQQ for preparing a feed additive in order to preserve the quality of meat obtained from poultry. In accordance with the disclosure, the meat is protected from the adverse effects of myopathies as previously described. Preferably, said farmed animals are prone to or affected by myopathy, such as breast myopathy, and in particular at least one of wooden breast, white stripping and spaghetti meat;

a method for preventing myopathies with poultry; in an embodiment of the disclosure, the method comprises administering pyrroloquinoline quinone (PQQ) to said poultry; in a further embodiment of the disclosure, the method comprises administering a feed additive comprising PQQ to said poultry. Preferably, said farmed animals are prone to or affected by myopathy, such as breast myopathy, and in particular at least one of wooden breast, white stripping and spaghetti meat;

pyrroloquinoline quinone (PQQ) for use in preventing the apparition of the defaults in meat, or to limit or even to stop the development of defaults in meat, said defaults being linked to myopathy, with farmed animals;

a feed additive for use for preventing the apparition of the defaults in meat, or to limit or even to stop the development of defaults in meat, said defaults being linked to myopathy myopathies with farmed animals, said feed additive comprising PQQ;

As previously mentioned, wooden breast is observed in fast growing poultry of at least around 2 kg, possibly up to 5 kg, the lesions of myopathy increasing with weight. In one embodiment, the present disclosure is used to prevent wooden breast in poultry.

The present disclosure further pertains to a feed additive for preventing breast myopathies in poultry comprising pyrroloquinoline quinone (PQQ). In one embodiment, the feed additive is for preventing the apparition of the defaults in meat, or to limit or even to stop the development of defaults in meat, said defaults being linked to myopathy myopathies.

Another aspect of the disclosure is a feed or a premix for preventing at least one of wooden breast, white stripping and spaghetti meat in poultry, comprising pyrroloquinoline quinone (PQQ) or comprising said feed additive. In one embodiment, the feed or the premix aims at preventing the apparition of the defaults in meat, or to limit or even to stop the development of defaults in meat, said defaults being linked to the above myopathies. In this aspect, a feed preferably comprises 0.1 to 2.0 ppm of PQQ, preferably 0.2 to 1 ppm of PQQ.

Further, in this aspect, a premix comprises an amount of PQQ such that when mixed with 1 kg of feed, it provides a feed of the disclosure as described above.

The method of the disclosure shows high performances in at least one of preventing, limiting and even stopping meat defaults said meat defaults being linked to myopathies in farmed animals.

The following examples and the FIGURE illustrate embodiments according to the disclosure described above, in poultry. Specifically, they provide an evaluation of the potential of PQQ alone or in combination with a feed supplement on the incidence of white stripping (WS) and woody breast (WB), when supplemented in growing and finishing broiler diets.

### EXAMPLE 1

Study Animals:
Animal: Broiler Chicken
Breed/Strain: Ross 708
Description: Day-of-hatch male chicks, sexed at hatchery
Sex: Male

Materials and Methods:

Animals Required:

Total number of treatments: 4

Total number of replicates per treatment: 12

Total number of chicks per replicate: 40

Initial age: day-of-hatch: 0 days-of-age

Final age: 7 weeks: 49 days-of-age.

Diets and Formulation:

Feed will be changed based on the days of each phase.
Starter feed will be weighed out and fed from day 0 to 14. On day 14, non-consumed starter feed will be weighed by pen and discarded.
Grower feed will be weighed, issued and fed from day 14 until 28. On day 28, non-consumed grower feed will be weighed by pen and discarded.
Finisher feed will be weighed, issued and fed from day 28 until 42. On day 42, non-consumed finisher feed will be weighed by pen and discarded.
Withdraw feed will be weighed, issued and fed from day 42 until 49. On day 49, non-consumed finisher feed will be weighed by pen and discarded.

Husbandry: Birds and their Management:

Mortality:

Mortality and culling records are recorded per pen. A cull is defined as a bird that cannot access the water.

Feed & Water:

Feed and water are provided ad libitum, and added to each pen as needed to maintain an acceptable feed level within the feeder and feed pan.

Temperature:

The birds are housed according to standard brooding and growing temperatures. The temperature outside of the house is recorded daily. Temperature and relative humidity within the house are recorded on a daily basis, at the chicks' level, in all phases of the grow-out.

Evaluated Parameters

Performance: Feed consumption and pen weights are determined on days 0, 14, 21, 28, 35, 42 and 49 to calculate average body weight (BW), body weight gain (BWG) and feed conversion ratio (FCR).

Final bird weights: At the end of the grow-out period (Day 49), all birds are weighed, as well as any and all remaining feed.

Processing: On day 49, ten (12) birds are selected from each pen for processing (at 12 reps/trt: 576 birds in total; 144 birds/trt).

Wooden breast assessment was determined by an expert panel according to 4 levels: No (WB1), Mild (WB2), Medium Severe (WB3), Severe (WB4).

Statistical Analyses

Randomized Complete Block Design with 4 treatments×12 replicates/treatment (40 birds/pen), randomized within blocks across the barn to minimize differences in environmental condition. The pen is the experimental unit.

Dietary Treatments
Control (NC, negative control without PQQ)
0.5 ppm PQQ
1 ppm PQQ
2 ppm PQQ

Effect of PQQ on the Body Weight (BW), Body Weight in (BWG), Feed Efficiency of Broilers and Feed Conversion Ratio (FCR)

The results are presented in the following tables 1-4 for broilers fed diets from the first week to the seventh week (corresponding to 0 to 49 days of age).

The values are means and SD is standard variation.

TABLE 1
Effect of PQQ on the body weight (Kg)

| | 0 ppm | | 0.5 PPM PQQ | | 1.0 ppm PQQ | | 2.0 ppm PQQ | |
|---|---|---|---|---|---|---|---|---|
| | mean | SD | mean | SD | mean | SD | mean | SD |
| wk 1 | 0.141 | 0.002 | 0.139 | 0.002 | 0.145 | 0.002 | 0.146 | 0.002 |
| wk 2 | 0.345 | 0.006 | 0.355 | 0.008 | 0.371 | 0.004 | 0.373 | 0.005 |
| wk 3 | 0.722 | 0.015 | 0.763 | 0.013 | 0.790 | 0.011 | 0.794 | 0.009 |
| wk 4 | 1.251 | 0.028 | 1.286 | 0.024 | 1.329 | 0.03 | 1.331 | 0.025 |
| wk 5 | 1.984 | 0.056 | 2.042 | 0.025 | 2.112 | 0.024 | 2.065 | 0.014 |
| wk 6 | 2.744 | 0.056 | 2.808 | 0.042 | 2.927 | 0.017 | 2.835 | 0.022 |
| wk 7 | 3.425 | 0.076 | 3.416 | 0.061 | 3.612 | 0.031 | 3.472 | 0.044 |

TABLE 2
Effect of PQQ on the average daily body weight gain (Kg)

| | 0 ppm | | 0.5 PPM PQQ | | 1.0 ppm PQQ | | 2.0 ppm PQQ | |
|---|---|---|---|---|---|---|---|---|
| | mean | SD | mean | SD | mean | SD | mean | SD |
| wk 1 | 0.095 | 0.002 | 0.095 | 0.002 | 0.100 | 0.002 | 0.102 | 0.002 |
| wk 2 | 0.205 | 0.005 | 0.215 | 0.007 | 0.225 | 0.004 | 0.224 | 0.004 |
| wk 3 | 0.393 | 0.01 | 0.414 | 0.005 | 0.419 | 0.008 | 0.425 | 0.005 |
| wk 4 | 0.547 | 0.014 | 0.556 | 0.010 | 0.573 | 0.012 | 0.548 | 0.012 |
| wk 5 | 0.671 | 0.021 | 0.688 | 0.011 | 0.723 | 0.009 | 0.727 | 0.013 |
| wk 6 | 0.760 | 0.014 | 0.767 | 0.018 | 0.787 | 0.007 | 0.77 | 0.015 |
| wk 7 | 0.680 | 0.029 | 0.622 | 0.028 | 0.686 | 0.023 | 0.648 | 0.030 |

The results of Tables 1 and 2 evidence a positive influence of PQQ on the body weight as from the first week with concentrations of 1 ppm and 2 ppm up to the end of the assay. For lower concentrations, precisely for a concentration of 0.5 ppm, a positive influence of PQQ is observed as from the second week.

TABLE 3
Effect of PQQ on the feed intake (Kg)

| | 0 ppm | | 0.5 PPM PQQ | | 1.0 ppm PQQ | | 2.0 ppm PQQ | |
|---|---|---|---|---|---|---|---|---|
| | mean | SD | mean | SD | mean | SD | mean | SD |
| wk 1 | 0.144 | 0.004 | 0.142 | 0.004 | 0.151 | 0.006 | 0.142 | 0.005 |
| wk 2 | 0.304 | 0.005 | 0.299 | 0.004 | 0.315 | 0.01 | 0.315 | 0.005 |
| wk 3 | 0.639 | 0.009 | 0.671 | 0.013 | 0.678 | 0.009 | 0.645 | 0.005 |
| wk 4 | 0.817 | 0.019 | 0.796 | 0.019 | 0.816 | 0.014 | 0.813 | 0.015 |
| wk 5 | 1.132 | 0.037 | 1.139 | 0.024 | 1.161 | 0.017 | 1.141 | 0.024 |
| wk 6 | 1.294 | 0.031 | 1.346 | 0.029 | 1.370 | 0.013 | 1.336 | 0.022 |
| wk 7 | 1.088 | 0.049 | 0.986 | 0.025 | 1.106 | 0.040 | 1.041 | 0.044 |

These results show that PQQ has an influence on the feed intake as from a concentration of 1 ppm.

TABLE 4
Effect of PQQ on feed conversion ratio (FCR)

| | 0 ppm | | 0.5 PPM PQQ | | 1.0 ppm PQQ | | 2.0 ppm PQQ | |
|---|---|---|---|---|---|---|---|---|
| | mean | SD | mean | SD | mean | SD | mean | SD |
| wk 1 | 1.518 | 0.048 | 1.491 | 0.047 | 1.514 | 0.067 | 1.406 | 0.056 |
| wk 2 | 1.489 | 0.036 | 1.400 | 0.037 | 1.418 | 0.059 | 1.414 | 0.027 |
| wk 3 | 1.632 | 0.030 | 1.623 | 0.030 | 1.619 | 0.015 | 1.608 | 0.011 |
| wk 4 | 1.503 | 0.047 | 1.435 | 0.035 | 1.427 | 0.018 | 1.469 | 0.032 |
| wk 5 | 1.694 | 0.046 | 1.617 | 0.013 | 1.608 | 0.032 | 1.605 | 0.039 |
| wk 6 | 1.701 | 0.024 | 1.755 | 0.021 | 1.741 | 0.019 | 1.741 | 0.034 |
| wk 7 | 1.603 | 0.038 | 1.603 | 0.042 | 1.616 | 0.039 | 1.615 | 0.027 |

The feed conversion ratio is the ratio between feed intake and body weight gain.

Effect of PQQ on Wooden Breast Proportions

For a more readable evidence of the benefic effects of the disclosure, the results are presented in the FIGURE.

### EXAMPLE 2

Study Animals:
Animal: Broiler Chicken
Breed/Strain: Cobb 500
Description: Day-of-hatch male chicks, sexed at hatchery
Sex: Male

Materials and Methods:

Animals Required:

Total number of treatments: 4

Total number of replicates per treatment: 12

Total number of chicks per replicate: 25

Initial age: day-of-hatch: 0 days-of-age

Final age: 6 weeks: 42 days-of-age.

Diets and Formulation:

Feed will be changed based on the days of each phase.
Starter feed will be weighed out and fed from day 0 to 7.
Grower feed will be weighed, issued and fed from day 8 until 21.
Finisher feed will be weighed, issued and fed from day 22 until 35.
Withdraw feed will be weighed, issued and fed from day 36 until 42.

Husbandry: Birds and their Management:

Mortality:

Mortality and culling records are recorded per pen. A cull is defined as a bird that cannot access the water.

Feed & Water:

Feed and water are provided ad libitum, and added to each pen as needed to maintain an acceptable feed level within the feeder and feed pan.

Temperature:

The birds are housed according to standard brooding and growing temperatures. The temperature outside of the house is recorded daily. Temperature and relative humidity within the house are recorded on a daily basis, at the chicks' level, in all phases of the grow-out.

Evaluated Parameters

Performance: Feed consumption and pen weights are determined on days 0, 21, 35 and 42 to calculate average body weight (BW), body weight gain (BWG) and feed conversion ratio (FCR).

Final bird weights: At the end of the grow-out period (Day 42), all birds are weighed, as well as any and all remaining feed.

Processing: On day 42, ten (12) birds are selected from each pen for processing (at 12 reps/trt: 576 birds in total; 144 birds/trt)

Wooden breast assessment was determined by an expert panel according to 5 levels: WB0, WB1, WB2, WB3, WB4 from no WB to severe WB.

Statistical Analyses

Randomized Complete Block Design with 4 treatments×12 replicates/treatment (40 birds/pen), randomized within blocks across the barn to minimize differences in environmental condition. The pen is the experimental unit.

Dietary Treatments
Control (NC, negative control without PQQ)
0.2 ppm PQQ
0.5 ppm PQQ
1 ppm PQQ

Effect of PQQ on the Body Weight Gain (BWG), Feed Efficiency of Broilers and Feed Conversion Ratio (FCR)

The results are presented in tables 5 to 13 below for broilers fed diets from 0 to 42 days (d) of age.

The values given are the mean and the P value.

TABLE 5
Effect of PQQ on cumulative body weight gain of broilers (g)

| | PQQ, ppm | 1 to 21 d | 1 to 35 d | 1 to 42 d |
|---|---|---|---|---|
| T1 | NC | 606 | 2116 | 3086 |
| T2 | 0.2 | 624 | 2125 | 3204 |
| T3 | 0.5 | 608 | 2123 | 3166 |
| T4 | 1.0 | 630 | 2178 | 3188 |
| P value | | 0.7687 | 0.0648 | 0.0954 |

The results of Table 5 evidence a positive influence of PQQ on the body weight with each concentration.

TABLE 6
Effect of PQQ on cumulative feed conversion ratio of broilers

| | PQQ, ppm | 1 to 21 d | 1 to 35 d | 1 to 42 d |
|---|---|---|---|---|
| T1 | NC | 1.349 | 1.494 | 1.724 |
| T2 | 0.2 | 1.355 | 1.494 | 1.674 |
| T3 | 0.5 | 1.402 | 1.526 | 1.675 |
| T4 | 1.0 | 1.429 | 1.540 | 1.708 |
| P value | | 0.6645 | 0.3753 | 0.2003 |

TABLE 7
Effect of PQQ on feed intake of broilers (g)

| | PQQ, ppm | 1 to 7 d | 8 to 21 d | 22 to 35 d | 36 to 42 d |
|---|---|---|---|---|---|
| T1 | NC | 158 | 659 | 2355 | 2120 |
| T2 | 0.2 | 158 | 677 | 2346 | 2218 |
| T3 | 0.5 | 156 | 699 | 2390 | 2105 |
| T4 | 1.0 | 159 | 747 | 2459 | 2113 |
| P value | | 0.6565 | 0.5034 | 0.2623 | 0.1843 |

TABLE 8
Cumulative feed intake of broilers (g)

| | PQQP, ppm | 1 to1 21 d | 11 to 35 d | 1 t1o 42 d |
|---|---|---|---|---|
| T1 | NC | 817 | 3163 | 5257 |
| T2 | 0.2 | 835 | 3173 | 5365 |
| T3 | 0.5 | 853 | 3236 | 5316 |
| T4 | 1.0 | 905 | 3357 | 5449 |
| P Value | | 0.5021 | 0.0663 | 0.5200 |

TABLE 9
Effect of PQQ on wooden breast means per treatment at 42 d

| | PQQ, ppm | WB |
|---|---|---|
| T1 | NC | 2.90^{a} |
| T2 | 0.2 | 2.92^{a} |
| T3 | 0.5 | 2.36^{b} |
| T4 | 1.0 | 2.26^{b} |
| P value | | <0.0001 |

Wooden Breast Scores were:
WB 0—normal breast; WB 1—mild hardening in the upper; WB 2—moderate hardening in the upper and/or lower part of the fillet; WB 3—severe hardening; WB 4—severe hardening with hemorrhagic lesions, increased volume, and presence of yellow fluid.

TABLE 10
Effect of PQQ on wooden breast myopathies scores at 42 d

| | PQQ, ppm | WB0 | WB1 | WB2 | WB3 | WB4 |
|---|---|---|---|---|---|---|
| T1 | NC | 0.00 | 0.33 | 1.42^{ab} | 2.67 | 1.58 |
| T2 | 0.2 | 0.08 | 0.17 | 1.17^{b} | 2.92 | 1.67 |
| T3 | 0.5 | 0.00 | 0.92 | 2.50^{ab} | 1.83 | 0.75 |
| T4 | 1.0 | 0.00 | 1.08 | 2.75^{a} | 1.50 | 0.58 |
| P value | | 0.554 | 0.051 | 0.021 | 0.080 | 0.126 |

Higher is the score per WB 3 and 4 categories, greater is the default. Conversely, for categories WB0 to WB2, higher is the score, better is the quality.

TABLE 11
Effect of PQQ on wooden breast myopathies
occurrence at 42 d (%)

| | PQQ, ppm | WB0 | WB1 | WB2 | WB3 | WB4 |
|---|---|---|---|---|---|---|
| T1 | NC | 0.00 | 5.56 | 23.62^{ab} | 44.44^{ab} | 26.39 |
| T2 | 0.2 | 0.83 | 2.78 | 20.00^{a} | 49.16^{a} | 27.22 |
| T3 | 0.5 | 0.00 | 15.29 | 41.67^{ab} | 30.56^{ab} | 12.50 |
| T4 | 1.0 | 0.00 | 18.05 | 46.94^{a} | 25.00^{b} | 10.00 |
| P value | | 0.537 | 0.052 | 0.022 | 0.036 | 0.150 |

Wooden breast scores were: WB0—normal breast; WB1—mild hardening in the upper; WB 2—moderate hardening in the upper and/or lower part of the fillet; WB 3—severe hardening; WB 4—severe hardening with hemorrhagic lesions, increased volume, and presence of yellow fluid.
Results show higher proportions of good quality breast fillets (WB0 to WB2) (+50% WB2 with 1 ppm) and lower proportions of bad quality fillets (WB3 and WB4) (−60% WB4 with 1 ppm) with supplementation with PQQ.

TABLE 12
Effect of PQQ on carcass and abdominal fat and yields
of the commercial cuts of broilers at 42 d (%)

| | | Abdominal | Breast | Breast | | | |
|---|---|---|---|---|---|---|---|
| PQQ, ppm | Carcass¹ | fat | tenders² | fillets | Thighs | Drumsticks | Wings |
| NC | 80.0^{a} | 1.10 | 5.00^{b} | 27.6 | 14.55^{ab} | 18.82^{ab} | 9.57^{b} |
| 0.2 | 80.3^{b} | 1.05 | 5.03^{b} | 26.8 | 14.36^{ab} | 18.42^{b} | 10.19^{ab} |
| 0.5 | 80.4^{b} | 1.11 | 5.12^{ab} | 26.8 | 15.01^{a} | 18.73^{ab} | 10.42^{a} |
| 1.0 | 80.3^{b} | 0.90 | 5.22^{a} | 27.4 | 14.24^{b} | 18.89^{a} | 10.05^{ab} |
| P-value | <0.0001 | 0.5190 | 0.0014 | 0.2147 | 0.0400 | 0.0026 | 0.0154 |

¹Eviscerated carcass as a percentage of body weight. whereas cuts are proportions of the carcass
²Skinless boneless Pectoralis major

TABLE 13
Effect of PQQ on carcass and abdominal fat and yields of the commercial cuts of broilers at 42 d (g)

| | | Abdominal | Breast | Breast | | | |
|---|---|---|---|---|---|---|---|
| Treatments | Carcass¹ | fat | tenders² | fillets | Thighs | Drumsticks | Wings |
| NC | 2.493 | 27 | 125b | 689 | 362 | 469 | 255 |
| 0.2 | 2.538 | 26 | 127ab | 680 | 364 | 467 | 258 |
| 0.5 | 2.527 | 28 | 129ab | 677 | 378 | 473 | 258 |
| 1.0 | 2.574 | 23 | 134a | 702 | 366 | 486 | 259 |
| P value | 0.2836 | 0.0480 | 0.0085 | 0.6638 | 0.1232 | 0.1414 | 0.9031 |

¹Eviscerated carcass as a percentage of body weight. whereas cuts are proportions of the carcass
²Skinless boneless Pectoralis major

1. Use of pyrroloquinoline quinone (PQQ) for preserving the quality of meat obtained from farmed animals.

2. Use of pyrroloquinoline quinone (PQQ) in at least one of preventing the apparition of the defaults in meat, limiting and stopping the development of defaults in meat, said defaults being linked to myopathy, with farmed animals.

3. Use of pyrroloquinoline quinone (PQQ) for preparing a feed additive or a premix in order to preserve the quality of meat obtained from farmed animals.

4. Use of pyrroloquinoline quinone (PQQ) for preparing a feed additive or a premix for at least one of preventing the apparition of the defaults in meat, limiting and stopping the development of defaults in meat, said defaults being linked to myopathy, with farmed animals.

5. The use of PQQ according to claim 1, wherein said farmed animals are prone to or affected by said myopathy.

6. The use of PQQ according to claim 1, wherein the farmed animals are poultry.

7. The use of PQQ according to claim 6, wherein said myopathy is selected from breast myopathies.

8. The use of PQQ according to claim 7, wherein said breast myopathy is selected from wooden breast, white stripping, and spaghetti meat.

9. The use of PQQ according to claim 1, wherein PQQ is liquid, granular, or in natural source.

10. The use of PQQ according to claim 1, wherein PQQ is in the form of granules that are mixed with a premix or a feed.

11. A feed additive for preserving the quality of meat obtained from farmed animals, said feed additive comprising pyrroloquinoline quinone (PQQ).

12. A feed additive for at least one of preventing the apparition of the defaults in meat, limiting and stopping the development of defaults in meat, said defaults being linked to myopathy, with farmed animals, said feed additive comprising pyrroloquinoline quinone (PQQ).

13. The feed additive according to claim 11, wherein PQQ is liquid or granular or in natural source.

14. The feed additive according to claim 11, wherein PQQ is in the form of granules that are mixed with a premix or a feed.

15. The feed additive according to claim 11, wherein the farmed animals are poultry.

16. The feed additive according to claim 15, wherein said myopathy is selected from breast myopathies.

17. The feed additive according to claim 16, wherein said breast myopathy is selected from wooden breast, white stripping and spaghetti meat.

18. A feed or the premix for preserving the quality of meat obtained from farmed animals, said feed comprising pyrroloquinoline quinone (PQQ) or a feed additive according to claim 10.

19. A feed or the premix for at least one of preventing the apparition of the defaults in meat, limiting and stopping the development of defaults in meat, said defaults being linked to myopathy, with farmed animals, said feed comprising pyrroloquinoline quinone (PQQ) or a feed additive according to claim 11.

20. The feed of claim 18, comprising 0.1-2.0 ppm of PQQ.

21. The feed of claim 20, comprising 0.2 to 1.0 ppm of PQQ.

22. The premix of claim 18, that when mixed with 1 kg of feed, provides a feed for preserving the quality of meat obtained from farmed animals, said feed comprising pyrroloquinoline quinone (PQQ) and said feed additive comprising pyrroloquinoline quinone.
